(19) United States
(12) Patent Application Publication
(10) Pub. No.: US 2006/0235352 A1
(43) Pub. Date: Oct. 19, 2006

(54) Gastric tube and method for introducing the same

(75) Inventors: Rainer Dziewas, Munster (DE); Salvador Peres-Mengual, Munster (DE); Peter Ludemann, Ahlen (DE)

Correspondence Address: FOLEY & LARDNER LLP, P.O. BOX 80278, SAN DIEGO, CA, 92138-0278, US

(73) Assignee: Westfalische Wilhelms-Universitat Munster, Munster (DE)

(21) Appl. No.: 10/535,790

(22) PCT Filed: Nov. 20, 2003
(86) PCT No.: PCT/DE03/03835

§ 371 Date: Apr. 7, 2006

(30) Foreign Application Priority Data
Nov. 21, 2002 (DE) 102 54 568.5

(51) Int. Cl. A61M 3/00

(52) U.S. Cl. 604/43

(57) ABSTRACT

The invention relates to a gastric tube for supplying or removing substances to or from the gastric tract of a patient, and to a method for introducing a gastric tube. In order to reduce the strain on the patient and the risk of injury, especially for patients with disturbed consciousness, the inventive gastric tube comprises a first tubular element (101, 201) which can be introduced into the gastric tract of a patient, forming a supply lumen (102) for substances to be supplied or removed to or from the gastric tract. Said first tubular element (101, 102) is formed from a skin-compatible and flexible material. The inventive gastric tube also comprises a second tubular element (108, 208) which is fixed to the first tubular element (101, 201) and provided with means for triggering the swallowing reflex of the patient, the second tubular element (108, 208) being more rigid than the first tubular element (101, 201).

The invention relates to a gastric tube and a method for introducing a gastric tube into the gastric tract of a patient.

A gastric tube serves, in particular, to supply and to remove substances into and from the gastric tract of a patient. If a patient suffering from a swallowing disturbance has to be fed artificially, food such as in the form of a nutriment solution can be supplied to the stomach of the patient by means of a gastric tube. Swallowing disturbances can particularly be caused when a patient is in a state of disturbed consciousness. For this reason, gastric tubes are in particular in such cases of major significance for artificial feeding.

Gastric tubes are additionally used for examination purposes in order to remove fully or partially the stomach contents of the patient for examination outside of the body.

In order to introduce the end of the gastric tube to be introduced into to the gastric tract, this end is introduced through the mouth (oral tube) or through the nose (nasogastric tube) and, supported by a swallowing action of the patient, is pressed forward into the oesophagus and, if required or desired, into the stomach of the patient. Especially where a prolonged dwell time of the tube is concerned, the nasogastric introduction is particularly suitable for this purpose.

A gastric tube according to the state of the art generally has two independent lumens, one of which is used for substance supply and removal, and the other for pressure equalisation by means of stomach ventilation.

Such a gastric tube is known for example from U.S. Pat. No. 5,643,230 and has a tubus that is divided into two independent lumens by means of a separating wall (one larger suction lumen for removing the stomach contents and a smaller ventilation lumen for pressure equalisation). A suction pump can be connected to the suction lumen at the extracorporal end of the tubus and at the end of the tubus to be introduced into the gastric tract, a flexible end piece with a plurality of longitudinally reaching ribs with interim-arranged suction openings is arranged, through which the stomach contents are sucked into the suction lumen in order to remove said contents for examination purposes.

From U.S. Pat. No. 5,560,747 it is for example also known to form the nasogastric tube for the realisation of several functions (e.g., pH-analysis etc.) with a multiple-channel configuration the flexible and compressible tubus having a plurality of passage channels spatially separated from one another. One of these passage channels forms an inner guide sleeve made of flexible and thermoplastic material for a nutriment supply tubus passed all the way through it.

When moving the gastric tube forward, there is in principle the danger that sensitive tissue is injured as a result of perforation caused by the gastric tube. In order to reduce the danger of injury, it is known from U.S. Pat. No. 5,700,252 to provide the tubus of the gastric tube, which is made from a relatively rigid material, with a flexible tip. As the tip bends upon deviation from the central forward movement line during the forward movement of the gastric tube, a guiding of the forward movement of the tubus in a direction away from the tissue wall is brought about by the bending action, and the risk of a perforation of the tissue is reduced in the process. From U.S. Pat. No. 5,334,167 it is known to pass the tubus all the way through a sleeve attached at the gastric tube circumferential side, which sleeve is made from polyvinyl chloride.

Where these gastric tubes are concerned, the danger of injury for patients with a normal state of consciousness can be reduced up to a certain degree. However, the assistance of the patient is often required in this case.

However, the gastric tubes as described above have the disadvantage that an automatic swallowing action of the patient during the introduction of the gastric tube is required. This fact considerably complicates the introduction of the gastric tube on its route into the gastric tract in patients with non-existent or restricted cooperation willingness or cooperation capability. As a result thereof, not only the strain on the patient substantially increases during the introduction of the tube but also the danger of injury to sensitive tissue as well. This is particularly the case with stroke patients both in the acute illness phase as well as in the rehabilitation phase because these patients are considerably restricted with regard to cooperation capability caused by reduced watchfulness and attentiveness (so-called vigilance minimisation), a loss of speech comprehension (aphasia) of the patient resulting from damage to the responsible brain sections of the patient, and/or the incapacity to perform specific movements (apraxia).

It is known in such cases to facilitate the introduction of the gastric tube by means of additional precautions such as, in particular, the visual inspection in the way of laryngoscopy with the use of a laryngoscope. These additionally deployed means, however, lead to a further strain on the patients and to an increase of the danger of injury.

It is therefore the task of this invention to provide a gastric tube and to establish a method for the introduction of a gastric tube into the gastric tract of a patient, through which the strain on the patient and the danger of injuries are reduced, particularly also for patients with a disturbed state of consciousness.

This task is solved by means of a gastric tube and a method for introducing a gastric tube in accordance with the features of the independent Claims.

A gastric tube for supplying and removing substances into and from the gastric tract of a patient has a first tubular element, which is introduced into the gastric tract of a patient under formation of a supply lumen for substances to be supplied to and removed from the gastric tract, or which is suitable for accommodating and introducing endoscopic instruments or surgical instruments. In the cases for supply and removal of substances, the first tubular element is formed from at least a partially flexible material that is preferably skin-compatible, particularly in respect of mucous membranes. The gastric tube also has a second tubular element, which is fixed to the first tubular element and is provided with means for triggering a swallowing reflex of the patient, the second tubular element having an increased rigidity relative to the first tubular element. The triggering of the swallowing reflex takes place preferably by means of physical stimulation of the throat wall, for example by loading with a small quantity of water.

The fixation of the second tubular element on the first tubular element can be in particular a detachable fixation, meaning, that means for the detachable fixation of the second tubular element at the first tubular element are preferably envisaged. These means can have, for example, an elastic plug placeable at the extracorporal end of the gastric tube between the first and second tubular element, e.g., made from caoutschouc material or similar, through which a detachable connection is established between the first and the second tubular element, and which can be removed in an uncomplicated manner, as also the second tubular element, after triggering the swallowing reflex and the introduction of the gastric tube into the gastric tract.

As an alternative to the above-mentioned fixation by means of an elastic plug, any other randomly selected means can be used for a detachable fixation of the second tubular element at the first tubular element.

In order to provide for a better detachability of the second tubular element after introducing the gastric tube into the gastric tract, the second tubular element can be coated also with a lubricant such as silicone oil.

The invention can also be used for applications where, for example, a tube for gastroscopy, echocardiography or oesophagoscopy is to be passed all the way through the supply lumen. Particularly in the case of the intended passing of a tube for oesophagoscopy through the supply lumen, there is no forward movement of the first and second tubular element all the way into the stomach, but instead only up to the oesophagus. In the sense of the invention, the gastric tract therefore comprises the oesophagus and the stomach.

For this reason, the invention also relates to a further design of a gastric tube for the introduction of endoscopic instruments into the oesophagus or stomach of a patient,
with a first tubular element (101, 201) in the form of an endoscopy tube that is preferably coated at least partially with a skin-compatible material or is formed from this; and
and a second tubular element (108, 208), which is fixed on the first tubular element (101, 201) and is provided with means for triggering a swallowing reflex of the patient.

With this embodiment a second tubular element is envisaged at an endoscopy tube, as it is known from the state of the art, which tubular element is fixated at the endoscopy tube or is integrated into this, and is provided with means for triggering the swallowing reflex of the patient. With this embodiment, the fixation can be in particular a detachable fixation also.

As a result of the second tubular element provided with means for triggering the swallowing reflex, the introduction of the gastric tube can be carried out with patients suffering from a disturbance of consciousness because no consciously exercised swallowing action is required from the patient for introducing the gastric tube all the way into the stomach. Subsequently, the introduction is possible also with patients suffering from a disturbance of consciousness with restricted or non-existent cooperation capability without any increase of the danger of injury while the gastric tube is being introduced.

As the first tubular element, which is used for forming a supply lumen and, subsequently, should have a comparably larger diameter, is formed preferably from a skin-compatible and flexible material, the danger of damage by perforation of the sensitive body tissue by means of this tubular element is minor.

At the same time, and as a result of the increased rigidity of the second tubular element which merely serves the purpose of triggering the swallowing reflex of the patient and therefore only needs a relatively small diameter, a good controllability of the gastric tube is ensured as the second tubular element is fixed on the first tubular element. In this way and with a careful introduction of the object into the stomach, a sufficient clearance between the gastric tube and the surrounding body tissue can be maintained without any problems.

The means for triggering the swallowing reflex preferably have a supply pump connected to the second tubular element for pumping a liquid all the way through the second tubular element. The supply pump is preferably a dosing pump for pumping a measured quantity of liquid all the way through the second tubular element, the measured volume of the supply pump being particularly and preferably adjustable in a range of (0.5-2.0) ml. With this defined adjusting capability, a particularly good reproducibility of the introducing action is ensured by the repetitive capability of an optimally set dosage quantity.

Preferably, the inner diameter of the second tubular element by means of capillary-type formation of the second tubular element is selected to such a minor size that the second tubular element is completely filled with liquid before the introduction operation as a result of its capillarity. In this way it is ensured that, after introducing the end of the gastric tube to be introduced into the gastric tract and after pushing forward this end up to the level of the throat rear wall below the palatine arches, the liquid for triggering the swallowing reflex is immediately supplied instead of air.

According to a preferred embodiment, the second tubular element is radially arranged on the inside of the first tubular element. In this case, particularly the first and the second tubular element can be in a coaxial arrangement to one another. In this way, the second tubular element with the larger rigidity is fully shielded off to the surrounding body tissue by the first and softer skin-compatible tubular element, so that the danger of injury is minimised.

According to a preferred embodiment, means for preventing the second tubular element from sliding out of the first tubular element during the introduction of the gastric tube into the gastric tract are envisaged.

These means can be, for example, a rubber ring solidly attached to the tubular element end to be introduced into the gastric tract on the inside of the first tubular element, or a thickening of the first tubular element at its end to be introduced into the gastric tract.

Furthermore, the second tubular element can also be preferably formed shorter than the first tubular element so that the second tubular element does not protrude from the first tubular element during the introduction of the gastric tube into the gastric tract with the end to be introduced into the gastric tract. For this purpose, the second tubular element is preferably shortened relative to the first tubular element up to the length which approximately corresponds to the outer diameter of the first tubular element.

According to another embodiment, the second tubular element can be fixed also at the outer periphery of the first tubular element in the longitudinal direction and parallel to this. This embodiment is particularly preferred especially when introducing endoscopic and/or surgical instruments, e.g., knives for removing tissue samples in the gastric tract, etc., because a safe triggering of the swallowing reflex is ensured in this way and the instruments can be introduced at the same time without problems.

According to a further preferred embodiment, the second tubular element has an increased Shore hardness (stipulated in DIN 53505) for the purpose of increasing the rigidity relative to the first tubular element. According to a further preferred embodiment, the second tubular element can be manufactured from a material whose Shore hardness is approximately 10% higher than the Shore hardness of that particular material from which the first tubular element is made. According to a further preferred embodiment, a thermoplastic or elastomer synthetic material with a Shore hardness A of 70-90, preferably about 80, can be used as a material for the first tubular element. As a material for the second tubular element, for example, a synthetic material such as hard-PVC with a Shore hardness D of approximately 40 to 60 can be used. Preferably, the second tubular element is also formed from a material which has such a low level of compressibility that no liquid escapes from the second tubular element during the introduction of the gastric tube, meaning, during “normal” handling of the same.

Alternatively or additionally to this, the second tubular element can have a plurality of reinforcement ribs arranged on the circumference for the purpose of increasing the rigidity relative also to the first tubular element.

According to a preferred embodiment, the first and/or the second tubular element is manufactured from an elastomer synthetic material, where the elastomer synthetic material can be a polycondensate, polymeride or polyaduct.

Furthermore, the first and/or second tubular element is preferably manufactured from a material which is selected from a group comprising polyurethane, polyamide, polyolefine or silicone.

According to a further preferred embodiment, the first and/or second tubular element is manufactured from a material to which an additive and/or a softener is added.

According to a further preferred embodiment, the elasticity of the first and/or second tubular element is controlled by way of the degree of polymerisation, the degree of cross-linkage and/or the degree of branching of the synthetic material used.

According to a further preferred embodiment, the second tubular element is manufactured from a preferably glass-fibre-reinforced compound synthetic material for the purpose of increasing the rigidity.

Alternatively or additionally, the second tubular element can also have a plurality of reinforcement ribs arranged on the circumferential side for the purpose of increasing the rigidity relative to the first tubular element.

According to a preferred embodiment, the first tubular element is rounded off or has a cone-shaped formation at its end to be introduced into the gastric tract, by means of which the danger of injury is further reduced.

Furthermore, the first tubular element can also be provided with markings on the outside which designate approximately the typically suitable insertion depth of the tube up to the point of triggering of the swallowing reflex.

For an effective triggering of the swallowing reflex with a relatively low dosage quantity the second tubular element has preferably on its end section, to be introduced into the gastric tract, a nozzle attachment for ejecting the quantity of liquid from the second tubular element with increased speed.

Preferably, the second tubular element has an outer diameter in the range of (0.8-1.2) mm, and the first tubular element has an outer diameter in the range of (3-5) mm.

According to a further preferred embodiment, a third tubular element is furthermore envisaged which forms a ventilation lumen for pressure relieving the stomach during the supply by way of the supply lumen. In this way, the occurrence of overpressure and underpressure in the stomach is effectively prevented when supplying and removing substances into and from the gastric tract of a patient.

The third tubular element can be preferably arranged in a radial manner on the inside, particularly coaxially to the first tubular element. In this way, the third tubular element is shielded off completely by the first soft and skin-compatible tubular element opposite the surrounding body tissue and the danger of injury is minimised as a result.

Preferably, the third tubular element is shorted relative to the first tubular element in the axial direction under formation of a projecting area of the first tube section over the end of the third tube section to be introduced into the gastric tract. With this configuration it is prevented that, during the suction operation of the third tubular element for pressure relieving the stomach, when supplying a nutrition solution, for example, to the stomach, the end of the third tubular element to be introduced into the gastric tract clings solidly by a suctioning effect to the stomach wall, because it is spaced from this as a result of the projecting area of the first tube section.

According to a further preferred embodiment, the first tubular element has air holes arranged on the circumference on the end section to be introduced into the gastric tract. With this configuration, the ventilation for pressure relieving the stomach interior can take place through the ventilation lumen of the third tubular element all the way through the air holes so that, when withdrawing air from the stomach interior by way of the ventilation lumen for the purpose of pressure equalisation, the danger of a seize-suction of the end of the gastric tube, to be introduced into the gastric tract, at the stomach wall is further reduced.

A method for introducing a gastric tube for the supply and removal of substances into and from the gastric tract of a patient has the following steps:
oral or transnasal introduction of a first tubular element formed from a skin-compatible and flexible material and a second tubular element fixed to the first tubular element and being more rigid than the first tubular element for allowing the forward movement into the gastric tract of the patient up to the level of the throat rear wall below the palatine arches of the patient;
triggering of the swallowing reflex of the patient by applying a preferable mechanical stimulation to the throat wall; and
further forward pushing of the first and second tubular element (101, 201, 108, 208) into the oesophagus, and if necessary into the stomach, in such a way that the second tubular element (108, 208) forms a supply lumen (102, 202) for substances to be supplied to or removed from the gastric tract, respectively.

For the external triggering of the swallowing reflex of the patient, and according to a preferred embodiment, a measured quantity of liquid is pumped all the way through the second tubular element in such a way that the measured quantity of liquid exits at the end, to be introduced into the gastric tract, at the level of the throat rear wall below the palatine arches.

Preferably, the second tubular element is filled fully with liquid at the beginning of the introduction operation. Distilled water, for example, can be used as a liquid.

According to a further preferred embodiment, the first and the second tubular elements are pushed forward all the way into the stomach of the patient in such a way that the supply lumen allows the supply and the removal of substances into and from the stomach of the patient.

According to a further preferred embodiment, and after introducing the end of the gastric tube to be introduced into the gastric tract into the stomach of the patient, a pressure equalisation is performed in the stomach by means of a gas withdrawal by way of a ventilation lumen formed by a third tubular element.

According to a further preferred embodiment, the first and the second tubular elements are pushed forward all the way into the gastric tract of the patient in such a way that the supply lumen allows the passage of a tube for gastroscopy, echocardiography or oesophagoscopy all the way through the supply lumen.

The invention is described in the following in greater detail based on embodiment examples shown in the attached Figures.

The Figures show the following:

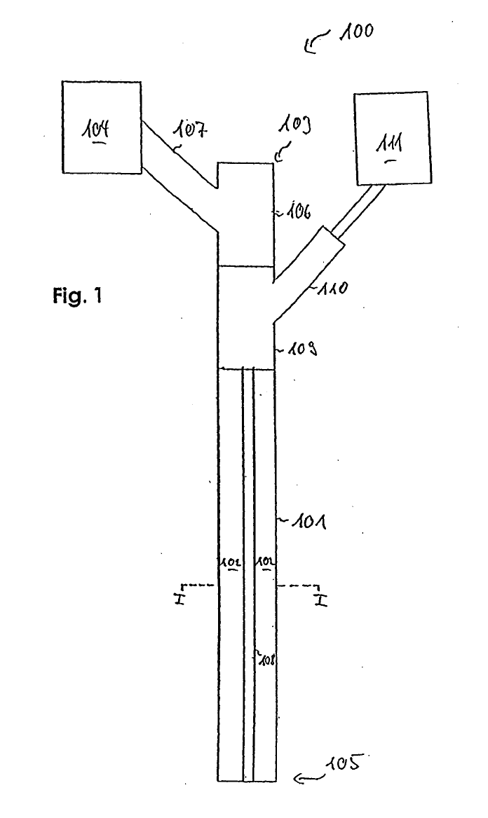

FIG. 1: a schematic side view of an inventive gastric tube according to a first preferred embodiment of the invention as presented here;

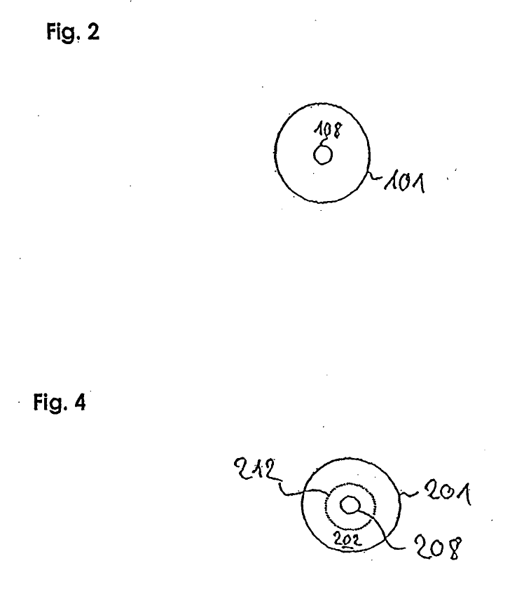

FIG. 2: a cross-sectional view of the gastric tube along the line I-I from FIG. 1.

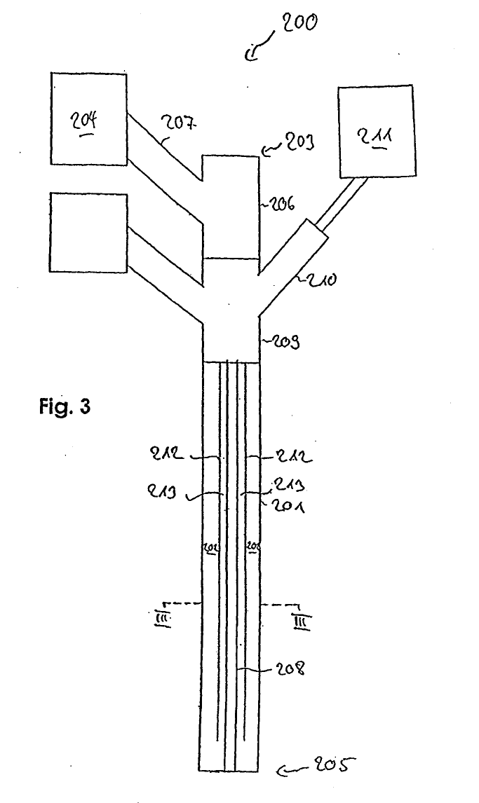

FIG. 3: a schematic side view of an inventive gastric tube according to a second preferred embodiment of the invention as presented here;

FIG. 4: a cross-sectional view of the gastric tube along the line III-III from FIG. 3.

According to FIG. 1, a gastric tube 100 according to a first embodiment example has a first tubular element 101 which forms a supply lumen 102. The first tubular element 101 is made from a skin-compatible and soft material, such as, for example, an elastomer synthetic material and has an outer diameter of 3-5 mm as well as a wall thickness of approximately (0.5-2.0) mm.

The supply lumen 102 reaches from an extracorporal end 103, where it is connected to a supply pump 104 for transporting substances to be supplied to and removed from the gastric tract, up to the end 105 of the gastric tube 100 to be introduced into the gastric tract, this said end can be pushed forward into the stomach of the patient. For connecting the supply pump 104 to the extracorporal end 103 of the gastric tube 100, a connecting piece 106 is attached, at the to the extracorporal end 103, with a branch-off 107 leading to the supply pump 104.

Radially on the inside of the first tubular element 101 as well as in a coaxial position to this, a second tubular element 108 is arranged and fixed to the first tubular element 101. The second tubular element 108 has an outer diameter of approximately (0.8-1.2) mm as well as a wall thickness of approximately (0.5-1.5) mm and, by way of a second connecting piece 109 arranged adjacent to the first connecting piece 106, which second connecting piece has a branch-off 110, is connected at the extracorporal end 106 of the gastric tube 100 to a dosing pump 111 in such a way that a liquid, preferably distilled water, can be pumped all the way through the second tubular element 108 up to the end 105 of the gastric tube 100 to be introduced into the gastric tract. The measuring volume of the dosing pump 111 is adjustable in a range of (0.5-2.0) ml.

According to an alternative but, however less preferred embodiment, also a supply pump without an adjustable dosage quantity or also a manually operable injection syringe can be used instead of the dosing pump 111. Furthermore, of course, the connecting piece 106 and the connecting piece 109 can also be formed in one piece as a single connecting piece with double branch-off, or the corresponding branch-offs leading to the supply pump 104 and the dosing pump 111, respectively, can be joined integrally with the first and second tubular element 101, 108, respectively.

In the operation of the gastric tube 100 according to the invention and according to the first embodiment example, the end 105 of the gastric tube 100 to be introduced into the gastric tract, meaning, both tubular elements 101 and 108 fixed to one another, is pushed forward through one nostril of the patient into the pharyngeal cavity until the end 105 to be introduced into the gastric tract is approximately located at the level of the throat rear wall below the palatine arches, which typically corresponds to an introduction length of approximately (11-13) cm. During this forward movement, the second tubular element 108 is preferably and already filled completely with the liquid (e.g. distilled water) supplied from the dosing pump. As soon as this position is attained, the measured volume set at the dosing pump of, for example, 0.5 ml distilled water is pumped by way of the dosing pump 111 into the second tubular element 108 so that the liquid exits at the end 105 to be introduced into the gastric tract and is sprayed onto the throat below the palatine arches. In this way, the swallowing reflex of the patient, which can be observed without a problem based on the characteristic movement of the larynx, is triggered, upon which the end 105 of the gastric tube 100 to be introduced into the gastric tract is pushed forward carefully all the way into the stomach of the patient.

A further embodiment example of a gastric tube 200 according to the invention is shown in FIGS. 3 and 4, the same or similar elements of the gastric tube 200 with corresponding reference signs being marked with reference to the gastric tube 200, so that a detailed explanation of such is waived in the continued description below.

The gastric tube 200 is structured according to the gastric tube 100 but has, in contrast to the gastric tube 100, and additional third tubular element 212, which is arranged in the radial direction between the first tubular element 201 and the second tubular element 208 as well as being coaxial to these. The third tubular element 212 forms a ventilation lumen 213 for the pressure relieving the stomach during supply by way of the supply lumen 202 so that, during supply and/or removal of substances into and from the stomach of the patient as a result of the pressure equalisation by way of the ventilation lumen, the occurrence of an overpressure or underpressure in the stomach is effectively prevented. For this purpose, the third tubular element 212 is connected at its extracorporal end to a ventilation pump 214. This is effected according to FIG. 3 by way of a further branch-off 215 of the connecting piece 209, where, of course, a separate connecting piece thereof or an integral formation with the third tubular element can be envisaged.

As can be seen from FIG. 3, the third tubular element 212 is shortened relative to the first tubular element 201 in the axial direction of the gastric tube 200 under formation of a projecting area of the first tube section 201 over the end of the third tube section 212 to be introduced into the gastric tract. In this way, a seize-suctioning effect of the end of the third tubular element 212 to be introduced into the gastric tract is prevented at the stomach wall if, for example, air is sucked up from the stomach by way of the ventilation lumen 213 for the purpose of pressure relieving the stomach during the supply of a nutriment solution.

For the purpose of facilitating the sucking up and/or the supply of air by way of the ventilation lumen 213, the first tubular element 201 furthermore has air holes (not shown) on the circumference of the end section to be introduced into the gastric tract, which air holes are preferably envisaged only in the projecting area of the first tube section 201 over the end of the third tube section 212 to be introduced into the gastric tract. In this way, the danger of a seize-suctioning of the end section of the gastric tube to be introduced into the gastric tract is further reduced.

The gastric tube according to the invention is preferably formed as a nasogastric tube. Possible in principle but, however, less preferred is also the formation as an oral tube when no prolonged dwell time is intended in particular and the gastric tube is to be applied for a brief examination only.

The introduction of the gastric tube according to the invention can also take place with patients suffering from a disturbance of consciousness with restricted or non-existent cooperation capability, without the danger of injury being increased during the introduction of the gastric tube because no consciously exercised swallowing action on the part of the patient is required for introducing the gastric tube all the way into the stomach.

As the first tubular element is formed from a skin-compatible and flexible material, the danger of damage by perforation of the sensitive body tissue by this tubular element is minor. At the same time, a good degree of controllability of the gastric tube is ensured by the increased rigidity of the second tubular element. In this way and with a careful introduction of the object into the stomach, sufficient distance can be maintained between the gastric tube and the surrounding body tissue without any problems, so that the danger of injury also with patients suffering from a disturbance of consciousness is minimised as a result.

### LIST OF REFERENCE SIGNS

100 Gastric tube
101 First tubular element
102 Supply lumen
103 Extracorporal end
104 Supply pump
105 End to be introduced into the gastric tract
106 Connecting piece
107 Branch-off
108 Second tubular element
109 Connecting piece
110 Branch-off
111 Dosing pump
200 Gastric tube
201 First tubular element
202 Supply lumen
203 Extracorporal end
204 Supply pump
205 End to be introduced into the gastric tract
206 Connecting piece
207 Branch-off
208 Second tubular element
209 Connecting piece
210 Branch-off
211 Dosing pump
212 Third tubular element
213 Ventilation lumen
214 Ventilation pump
215 Branch-off

1. A gastric tube for the supply or removal of material into and out of the oesophagus or the stomach of a patient comprising:
a) a first tubular element adapted to be introduced into the stomach of a patient, said first tubular element comprising a lumen to supply or remove material or endoscopic instruments, wherein the first tubular element is at least partially formed from flexible material; and
b) a second tubular element which is fixed to the first tubular element; said second tubular element comprising a lumen connected with a device for triggering a swallowing reflex of the patient, wherein the second tubular element has an increased rigidity relative to the first tubular element.

2. The gastric tube according to claim 1, said device for for triggering the swallowing reflex comprises a supply pump connected to the lumen of the second tubular element for pumping a liquid through the second tubular element lumen.

3. The gastric tube according to claim 2, wherein the supply pump is a dosing pump for pumping a measured quantity of liquid through the second tubular element.

4. The gastric tube according to claim 3, wherein the dosage quantity of the dosing pump is in a range of 0.5-2.0 ml.

5. The gastric tube according to claim 1 wherein the first or second tubular element is manufactured from an elastomeric synthetic material.

6. The gastric tube according to claim 5, wherein the elastomeric synthetic material is a polycondensate, polymeride or polyaduct.

7. The gastric tube according to claim 6, wherein the first or second tubular element is manufactured from a material which comprises polyurethane, polyamide, polyolefine or silicone.

8. The gastric tube according to claim 5, wherein the first or second tubular element is manufactured from a material to which a softener is added.

9. The gastric tube according to any one of the claim 5, wherein the elasticity of the first or second tubular element is controlled by way of the degree of polymerisation, the degree of cross-linkage and/or the degree of branch-off of the synthetic material used.

10. The gastric tube according to claim 1, wherein the second tubular element has an increased Shore hardness for increasing the rigidity relative to the first tubular element.

11. The gastric tube according to claim 10, wherein the first tubular element having has a Shore hardness A in the range of 70 to 90 and the second tubular element has a Shore hardness D in the range of 40 to 60.

12. The gastric tube according to claim 1, wherein the second tubular element is manufactured preferably from a glass-fibre-reinforced compound synthetic material for the purpose of increasing the rigidity.

13. The gastric tube according to claim 1, wherein the second tubular element has a plurality of circumferentially arranged reinforcement ribs for the purpose of increasing the rigidity relative to the first tubular element.

14. The gastric tube according to claim 1, wherein said second tubular element is detachably fixed to said first tubular element.

15. The gastric tube according to claim 1, wherein the second tubular element at its end section to be introduced into the gastric tract, has a nozzle attachment for ejecting the quantity of liquid from the second tubular element with increased speed.

16. The gastric tube according to claim 1, wherein the lumen of the second tubular element is a capillary such that the second tubular element lumen can be completely filled with liquid prior to introduction of the gastric tube into the patient.

17. The gastric tube according to claim 1, wherein the second tubular element is arranged radially on the inside of the first tubular element.

18. The gastric tube according to claim 17, wherein the first and the second tubular element are each arranged coaxially to each other.

19. The gastric tube according to claim 17, wherein said second tubular element is fixed to said first tubular element by adapting the end of the first tubular element to prevent the second tubular element from sliding out of the end of the first tubular element.

20. The gastric tube according to claim 1, wherein said second tubular element is fixed parallel in the longitudinal direction at the outer periphery of the first tubular element.

21. The gastric tube according to claim 1, wherein the second tubular element has an outer diameter in the range of 0.8-1.2 mm and a wall thickness in the range of 0.5-1.5 mm.

22. The gastric tube according to claim 1, wherein the first tubular element has an outer diameter in the range of 3-5 mm and a wall thickness in the range of 0.5-2.0 mm.

23. The gastric tube according to claim 1, wherein the first tubular element is rounded off or has a conical shape at its end to be introduced into the gastric tract.

24. The gastric tube according to claim 1, wherein the first tubular element has at least one marking on the outside for designating the desired insertion depth up to the triggering of the swallowing reflex.

25. The gastric tube according to claim 1, further comprising a third tubular element fixed to the first or second tubular element, which forms a ventilation lumen for relieving stomach pressure during a supply by way of the supply lumen.

26. The gastric tube according to claim 25, wherein the third tubular element is arranged radially on the inside of the first tubular element.

27. The gastric tube according to claim 26, wherein the third tubular element is shorter relative to the first tubular element in the axial direction under formation of a projecting area of the first tube section over the end of the third tube section to be introduced into the gastric tract.

28. The gastric tube according to claim 1, wherein the first tubular element has air holes arranged on the circumference at its end section to be introduced into the gastric tract.

29. The gastric tube according to claim 1, wherein the supply lumen is for the supplying or removal of substances being formed in the stomach of the patient.

30. The gastric tube according to claim 1, wherein the supply lumen is formed in such a way that a tube for gastroscopy, echocardiography or oesophagoscopy can be passed through the supply lumen.

31. A method for introducing a gastric tube for the supply or removal of substances or endoscopic instruments into or out of the stomach of a patient, the method comprising:
a) inserting into the oral or nasal cavity up to the level of the throat rear wall below the palatine arches of the patient a first tubular element formed from a flexible material and a second tubular element fixed to the first tubular element, said second tubular element being more rigid than the first tubular element adapted for inserting;
b) triggering the swallowing reflex of the patient; and
c) continuing to insert said first and second tubular element into the oesophagus, and into the stomach as required, in such a way that the second tubular element forms a supply lumen for substances to be supplied to or removed from the gastric tract.

32. The method according to claim 31, further comprising pumping a measured quantity of liquid through the second tubular element for the purpose of triggering the swallowing reflex of the patient, so that the measured quantity of liquid exits at the end, to be introduced into the gastric tract, at the level of the throat rear wall below the palatine arches.

33. The method according to claim 32, wherein the second tubular element is completely filled with liquid prior to introduction of the first tubular element.

34. The method according to claim 32 or 33, wherein the liquid is distilled water.

35. The method according to claim 31, further comprising performing pressure equalization in the stomach by gas withdrawal by way of a ventilation lumen formed by a third tubular element after introducing the end of the gastric tube into the gastric tract into the stomach of the patient.

36. The gastric tube for the supply of endoscopic instruments into the oesophagus or the stomach of a patient, comprising;
a) a first tubular element comprising a flexible material and formed in the shape of an endoscopy tube; and
b) a second tubular element fixed to the first tubular element and connected to a device for triggering the swallowing reflex of the patient.

37. The gastric tube according to claim 36, wherein said device for triggering the swallowing reflex has a supply pump connected to the second tubular element for pumping a liquid all the way through the second tubular element.

38. The gastric tube according to claim 37, wherein the supply pump is a dosing pump for pumping a measured quantity of liquid through the second tubular element.

39. The gastric tube according to claim 38, wherein the dosage quantity of the dosing pump adjustable and in a range of 0.5-2.0 ml.

40. The gastric tube according to claim 1, 31 or 36, wherein the first tubular element is coated with a skin-compatible material.

41. The method according to claim 31, wherein triggering is by physical stimulation of the throat wall.

42. The gastric tube according to claim 17 or 18, wherein the second tubular element is fixed to the first tubular element by a retaining element.
